(19) United States
(12) Patent Application Publication
(10) Pub. No.: US 2004/0265256 A1
(43) Pub. Date: Dec. 30, 2004

(54) Alkaline hair-relaxing in the presence of at least one high molecular weight water-soluble polymer

(76) Inventors: Henri Samain, Bievres (FR); Aude Livoreil, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/837,642

(22) Filed: May 4, 2004

Related U.S. Application Data
(60) Provisional application No. 60/477,347, filed on Jun. 11, 2003.

(30) Foreign Application Priority Data
May 5, 2003 (FR) 03 05451

(51) Int. Cl. A61K 7/09; A45D 7/04

(52) U.S. Cl. 424/70.2

(57) ABSTRACT

A multi-component alkaline hair-relaxing agent comprising:

a first component (a) comprising at least one alkaline hair-relaxing composition, and

a second component (b) comprising at least one composition chosen from compositions comprising a dispersion of particles of at least one water-soluble polymer having a weight-average molecular mass of greater than 10⁶ in a saline aqueous solution, obtained by heterogeneous free-radical polymerization of water-soluble monomers with precipitation of the formed polymer and compositions comprising an aqueous solution of at least one water-soluble polymer with a weight-average molecular mass of greater than 10⁶, obtained by heterogeneous free-radical polymerization of water-soluble monomers with precipitation of the formed polymer, wherein the two components (a) and (b) may be applied simultaneously or successively to the keratin fibers to be relaxed; a ready-to-use hair-relaxing composition comprising the two components, and a hair-relaxing process using the multi-component alkaline hair-relaxing agent or the ready-to-use composition.

This application claims benefit of U.S. Provisional Application No. 60/477,347, filed Jun. 11, 2003.

Disclosed herein is the use of at least one water-soluble polymer having a high molecular weight and capable of protecting keratin fibers against chemical degradation during alkaline relaxing of the hair. Further disclosed herein is a multi-compartment kit for alkaline hair-relaxing comprising a dispersion and/or a solution of such a polymer, a ready-to-use hair-relaxing composition, and an alkaline hair-relaxing process using the kit or the ready-to-use composition.

The known processes for relaxing or straightening hair are based on two types of chemical treatment:

treatment of keratin fibers with a reducing agent, causing cleavage of the disulphide bridges (S—S) of the keratin fibers, followed by an oxidizing treatment applied to keratin fibers subjected to a mechanical constraint, and

treatment of keratin fibers subjected to a mechanical constraint with a strong alkaline agent.

These two types of treatment may efficiently relax curls, but may have at least one drawback. For example, the treatments may be very aggressive and may modify the chemical nature of the keratin fibers.

Repetition of these treatments may cause at least one of the following poor cosmetic hair properties: difficulty in disentangling, an unpleasant feel, coarse, dull hair, and, for example, degradation of the keratin fibers.

The degradation of the fibers is undesirable, for example, because it may irreversibly deteriorate at least one physicochemical hair property. For example, the hair may become more porous and consequently may be more difficult to dry. The hair may show greater sensitivity to other hair treatments, such as dyeing, and at least one mechanical or surface property may be unfavorably modified, which may be reflected, for example, by a reduction in the tensile breaking strength or an increase in the coefficient of friction.

The present inventors have discovered that it may be possible to significantly reduce the chemical degradation of keratin fibers resulting from alkaline hair-relaxing treatments, by using an alkaline hair-relaxing agent in combination with at least one polymer chosen from high molecular weight polymers, obtained by heterogeneous free-radical polymerization of water-soluble monomers with precipitation of the formed polymer in the reaction medium.

Surprisingly, the protective effect on the keratin fibers was observed by the inventors only for the alkaline hair-relaxing process, but was not observed for a process for relaxing hair via reduction/oxidation of the keratin fibers.

The protective effect on the keratin fibers may be obtained not only in the case of the joint use (simultaneous application) of the alkaline hair-relaxing composition and the high molecular weight polymer, but also when a two-stage hair-relaxing process is used. This process comprises treating the keratin fibers with the high molecular weight polymer followed by alkaline hair-relaxing, these two steps possibly being separated by an intermediate rinsing step. A smaller protective effect may be obtained when the high molecular weight polymer is applied after the alkaline hair-relaxing step.

The polymers disclosed herein are obtained by heterogeneous free-radical polymerization of a blend of water-soluble monomers. The polymerization medium is a saline aqueous solution of relatively high ionic strength, chosen such that the polymer, which is soluble in pure water, precipitates gradually as it is formed so as to form a dispersion of fine polymer particles in a continuous saline aqueous phase. This dispersion may have the advantage of having both a relatively high concentration of high molecular weight polymer and a relatively moderate viscosity, which may allow quick and easy mixing of the dispersion with the alkaline hair-relaxing composition. The latter property may allow the use of dispersions of a high molecular weight water-soluble polymer in hair-relaxing kits in which the alkaline composition and the water-soluble polymer may be packaged in separate compartments and mixed together immediately before being applied to the hair, or may be applied to the hair in succession at the time of use.

Thus, disclosed herein is a multi-component alkaline hair-relaxing agent comprising:

a first component (a) comprising at least one alkaline aqueous hair-relaxing composition, and

a second component (b) comprising at least one composition chosen from

compositions comprising a dispersion of particles of at least one water-soluble polymer, for example, a polyelectrolyte, having a weight-average molecular mass of greater than 10⁶ in a saline aqueous solution, obtained by heterogeneous free-radical polymerization of water-soluble monomers with precipitation of the formed polymer, and

compositions comprising an aqueous solution of at least one water-soluble polymer with a weight-average molecular mass of greater than 10⁶, obtained by heterogeneous free-radical polymerization of water-soluble monomers with precipitation of the formed polymer,

wherein the two components (a) and (b) are applied simultaneously or successively to the keratin fibers to be relaxed.

The multi-component alkaline hair-relaxing agent disclosed herein may, for example, be in the form of a multi-compartment kit for the alkaline relaxing of keratin fibers, comprising at least one first compartment comprising the first component (a) and at least one second compartment comprising the second component (b).

As indicated above, the synthesis of the at least one water-soluble polymer disclosed herein for protecting keratin fibers against alkaline degradation, is performed by heterogeneous free-radical polymerization of water-soluble monomers comprising at least one ethylenic unsaturation. The polymerization is performed in an aqueous solution of a mineral electrolyte (salt) having an ionic strength that is sufficient to cause the formed polymer to precipitate as soon as the at least one water-soluble polymer has reached a certain molecular mass. This polymerization technique thus makes it possible, by virtue of the well-known phenomenon of salting out, to prepare saline aqueous dispersions of water-soluble polymer particles. The polymer particles thus synthesized are distinguished by a high weight-average molecular mass, for example, of greater than 10⁶.

The technique of heterogeneous free-radical polymerization in an aqueous medium with precipitation of the formed polymer is described, for example, in Pat. No. U.S. Pat. No. 4,929,655, in Patent Application No. EP 0 943 628 and in International Patent Application No. WO 02/34796, the disclosures of which relating to this technique are incorporated herein by reference.

To ensure the stability of the polymer particle dispersions during synthesis and storage, the polymerization may be performed in the presence of at least one dispersant. The at least one dispersant may, for example, be chosen from polyelectrolytes, which, in contrast to the at least one water-soluble polymer, is soluble in the high ionic strength aqueous polymerization medium.

The at least one dispersant may, for example, have a charge identical to that of the at least one water-soluble polymer. In other words, for the synthesis of cationic polyelectrolytes, a cationic dispersing polyelectrolyte may be used.

In one embodiment, the at least one dispersant may be chosen from cationic polyelectrolytes obtained by polymerization of 50 mol % to 100 mol % of at least one cationic monomer chosen from salts, such as hydrochlorides and sulphates of dimethylaminoethyl (meth)acrylate, of N-dimethylaminopropyl(meth)acrylamide and of di(meth)allylamine, (meth)acryloyloxyethyltrimethylammonium chloride, (meth)acrylamidopropyltrimethylammonium chloride and dimethyldiallylammonium chloride, and from 50 mol % to 100 mol % of acrylamide. In one embodiment, a polyamine such as a polyalkyleneamine may also be used.

The at least one dispersant may, for example, be used in an amount ranging from 1% to 10% by weight, relative to the total weight of the monomers to be polymerized.

The saline aqueous solution that serves as the synthesis and dispersion medium for the at least one water-soluble polymer is a solution comprising at least one mineral salt, for example, chosen from divalent anionic salts. Examples of divalent anionic salts include ammonium sulphate, ammonium hydrogen sulphate, sodium sulphate, sodium hydrogen sulphate, magnesium sulphate, magnesium hydrogen sulphate, aluminium sulphate, and aluminium hydrogen sulphate. In one embodiment, the at least one mineral salt may be chosen from ammonium sulphate and sodium sulphate.

The at least one mineral salt may be present in an amount sufficient to result in the precipitation of the at least one water-soluble polymer formed in the polymerization medium. To obtain such a precipitation, the at least one mineral salt may be present in an amount at least equal to 15% by weight, relative to the total weight of the saline aqueous solution and may be present in an amount up to the saturation concentration of each salt. In one embodiment, the saline aqueous solution may also comprise at least one monovalent salt, for example, chosen from sodium chloride and ammonium chloride.

The heterogeneous free-radical polymerization in an aqueous medium as disclosed herein may be accompanied by a large increase in the viscosity of the reaction medium, which is reflected by at least one of the following difficulties: difficulty in stirring, lack of homogeneity of the reaction medium, and a broadening of the size distribution of the formed polymer particles. To prevent such an increase in viscosity, it has been proposed in European Patent Application No. 0 943 628 to add to the polymerization medium at least one agent for preventing the increase in viscosity of the reaction medium during polymerization.

The at least one water-soluble polymer disclosed herein may, for example, be prepared in the presence of the at least one agent for preventing the increase in viscosity.

The at least one agent for preventing the increase in viscosity of the reaction medium may be chosen, for example, from:

(A) polycarboxylic acids and salts thereof;

(B) polyphenols;

(C) cyclic compounds comprising at least one group chosen from hydroxyl and carboxyl groups, and salts thereof;

(D) gluconic acid and salts thereof;

(E) the reaction products obtained by reacting at least one monomer chosen from methoxyhydroquinone and cationic (meth)acrylic monomers with at least one radical-generating compound, under an oxidizing atmosphere;

(F) the reaction products obtained by reacting a cationic (meth)acrylic polymer with at least one radical-generating compound, under an oxidizing atmosphere; and

(G) the reaction products obtained by reacting a cationic (meth)acrylic polymer with at least one oxidizing agent.

The addition of at least one agent for preventing the increase in viscosity may allow the polymerization of the water-soluble monomers disclosed herein to be performed with a low-power stirrer while at the same time may avoid the formation of coarse particles. The at least one agent for preventing the increase in viscosity may, for example, be soluble in the aqueous reaction medium.

Examples of compounds (A) that may be used include oxalic acid, adipic acid, tartaric acid, malic acid, phthalic acid, and salts thereof.

Examples of compounds (B) that may be used include resorcinol and pyrogallol.

Examples of compounds (C) that may be used include m-hydroxybenzoic acid, p-hydroxybenzoic acid, salicylic acid, gallic acid, tannic acid, and acidic salts thereof.

Examples of compounds (D) that may be used include sodium gluconate, potassium gluconate, ammonium gluconate, and various amine salts of gluconic acid.

Examples of reaction products (E) that may be used include those obtained by reacting at least one radical-generating compound, under a stream of oxygenated gas, in a solution comprising at least one monomer chosen from methoxyhydroquinone and cationic (meth)acrylic monomers. The at least one radical-generating compound may be chosen from initiators commonly used for free-radical polymerization. Examples of the at least one radical-generating compound include water-soluble azo initiators such as 2,2′-azobis(2-amidinopropane) hydrochloride, sold, for example, under the name V-50 by the company Wako Chemical Industries, 2,2′-azobis[2-(2-imidazolin-2-yl)propane] hydrochloride, sold, for example, under the trade name VA-044 by the company Wako Chemical Industries, and an initiator from the group of water-soluble redox agents, such as the ammonium persulphate/sodium hydrogen sulphite combination.

Examples of reaction products (F) that may be used include those obtained by reacting at least one free-radical initiator, under an oxygenated atmosphere, with the at least one dispersant disclosed herein. The at least one free-radical initiator may be chosen from water-soluble azo initiators and water-soluble redox agents as disclosed herein.

Examples of reaction products (G) that may be used include those that may be obtained in the form of oxidized polymers of low molecular mass by oxidizing the at least one cationic dispersant disclosed herein obtained by polymerization of cationic (meth)acrylic monomers, using hydrogen peroxide or halogen as the at least one oxidizing agent.

The cationic (meth)acrylic monomers used for the preparation of the at least one agent for preventing the increase in viscosity (E), (F) and (G) may be chosen from dimethylaminoethyl (meth)acrylate hydrochloride and sulphate, (meth)acryloyloxyethyltrimethylammonium chloride, (meth)acryloyloxyethyldimethylbenzylammonium chloride, the hydrochloride and sulphate derived from N-dimethylaminopropyl(meth)acrylamide, (meth)acrylamidopropyltrimethylammonium chloride, dimethylaminohydroxypropyl (meth)acrylate chloride and sulphate, (meth)acryloyloxyhydroxypropyltrimethylammonium chloride and (meth)acryloyloxyhydroxypropyldimethylbenzylammonium chloride.

The at least one agent for preventing the increase in viscosity (A) to (G) may be used, alone or as a mixture, in an amount ranging from 10 ppm to 10 000 ppm by weight, relative to the total weight of the reaction solution.

The water-soluble monomers polymerized by heterogeneous free-radical polymerization in order to obtain the at least one water-soluble polymer are chosen from monomers comprising at least one ethylenic double bond, for example, chosen from vinyl, acrylic and allylic double bonds. The water-soluble monomers may be chosen from at least one of cationic, anionic and nonionic monomers.

The water-soluble anionic monomers may be chosen from acrylic acid, methacrylic acid, acrylamido-2-methylpropanesulphonic acid and itaconic acid. The water-soluble anionic monomers may be at least partially neutralized in the form of an alkali metal salt, an alkaline-earth metal salt, an ammonium salt, or a salt of an organic amine such as an alkanolamine.

The water-soluble nonionic monomers may be chosen from acrylamide, methacrylamide, N-vinylformamide, N-vinylacetonamide, hydroxypropyl acrylate, and hydroxy-propyl methacrylate.

The water-soluble cationic monomers may, for example, be chosen from di(C₁₋₄ alkyl)diallylammonium salts and the compounds of formula (I)

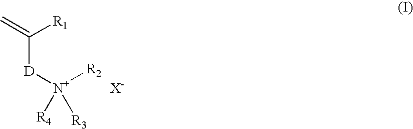

wherein:

R₁ is chosen from a hydrogen atom and a methyl group;

R₂ and R₃, which may be identical or different, are each chosen from a hydrogen atom and linear and branched C₁₋₄ alkyl groups;

R₄ is chosen from a hydrogen atom, linear and branched C₁₋₄ alkyl groups, and aryl groups;

D is chosen from groups of the following formula:

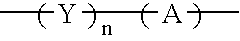

wherein:

Y is chosen from amide (—CO—NH—), ester (—O—CO— or —CO—O—), urethane (—O—CO—NH—), and urea (—NH—CO—NH—) functional groups;

A is chosen from linear, branched and cyclic C₁₋₁₀ alkylene groups, which may be substituted or interrupted with at least one ring chosen from divalent aromatic and heteroaromatic rings, and which may be interrupted with at least one hetero atom chosen from O, N, S and P, and which may comprise at least one functional group chosen from ketone, amide, ester, urethane, and urea functional groups;

n is a number equal to 0 or 1; and

X⁻ is chosen from anionic counterions, such as a chloride ion and a sulphate ion.

Examples of water-soluble cationic monomers include dimethylaminoethyl (meth)acrylate hydrochloride and sulphate, (meth)acryloyloxyethyltrimethylammonium chloride, (meth)acryloyloxyethyldimethylbenzylammonium chloride, N-dimethylaminopropyl(meth)acrylamide hydrochloride and sulphate, (meth)acrylamidopropyltrimethylammonium chloride, (meth)acrylamidopropyldimethylbenzylammonium chloride, dimethylaminohydroxypropyl (meth)acrylate hydrochloride and sulphate, (meth)acryloyloxyhydroxypropyltrimethylammonium chloride, (meth)acryloyloxyhydroxypropyidimethylbenzylammonium chloride, and dimethyldiallylammonium chloride.

The at least one water-soluble polymer may, for example, be chosen from polyelectrolytes, for example, cationic and amphoteric polyelectrolytes, i.e., a polyelectrolyte polymerized from at least one cationic monomer of formula (I).

In one embodiment of the multicomponent hair-relaxing agent disclosed herein, the at least one water-soluble polymer is obtained by heterogeneous free-radical polymerization of a monomer blend comprising from 0 to 30 mol % of acrylic acid, from 0 to 95.5 mol % of acrylamide, and from 0.5 mol % to 10 mol % of at least one cationic monomer of formula (I).

In another embodiment, the at least one water-soluble polymer is obtained by polymerization of a monomer blend comprising acrylic acid and at least one cationic monomer of formula (I), wherein the number of moles of the at least one cationic monomer of formula (I) is greater than the number of moles of acrylic acid.

The at least one water-soluble polymer may, for example, be chosen from those polymerized from monomer blends comprising, respectively:

10 mol % of acryloyloxyethyldimethylbenzylammonium chloride and 90 mol % of acrylamide;

30 mol % of acryloyloxytrimethylammonium chloride, 50 mol % of acryloyloxyethyldimethylbenzylammonium chloride and 20 mol % of acrylamide;

10 mol % of acryloyloxyethyltrimethylammonium chloride and 90 mol % of acrylamide; and

30 mol % of diallyldimethylammonium chloride and 70 mol % of acrylamide.

The at least one water-soluble polymer may also be chosen from anionic polyelectrolytes polymerized from a blend of anionic monomers and nonionic monomers. Examples of anionic polyelectrolytes include a copolymer obtained from a blend of 30 mol % of acrylic acid and 70 mol % of acrylamide.

The at least one water-soluble polymer disclosed herein may have a weight-average molecular mass of greater than 1 000 000 g/mol, for example, ranging from 1 000 000 to 50 000 000 g/mol. The weight-average molecular mass is determined by the RSV (Reduced Specific Viscosity) method as defined in “Principles of Polymer Chemistry”, Cornell University Press, Ithaca, N.Y., 1953, Chapter VII entitled “Determination of Molecular Weight”, pages 266-316.

The at least one water-soluble polymer in the dispersion or in the solution may be present in an amount ranging from 0.01 % to 20% by weight, relative to the total weight of the dispersion or solution.

The at least one alkaline hair-relaxing composition used in the multi-component alkaline hair relaxing agent is an aqueous composition comprising, in dissolved form, at least one hydroxide chosen from mineral and organic hydroxides, for example, chosen from hydroxides of an alkali metal, hydroxides of an alkaline-earth metal, hydroxides of a transition metal, hydroxides of metals from groups III, IV, V and VI, lanthanide and actinide hydroxides, ammonium hydroxides, and guanidinium hydroxide.

The at least one hydroxide may be formed in situ, for example, guanidine hydroxide may be formed by reacting calcium hydroxide and guanidine carbonate.

The at least one alkaline hair-relaxing composition may be present in an amount that is sufficient to allow the permanent reshaping of the treated keratin fibers. Such a permanent reshaping may be obtained when the at least one alkaline hair-relaxing composition has a pH ranging from 9 to 13 and, for example, from 10 to 13. The at least one alkaline hair-relaxing composition may also comprise at least one additive chosen from those that are common in hair cosmetics, for example, fatty substances.

One embodiment disclosed herein relates to a ready-to-use hair-relaxing composition comprising at least one water-soluble polymer with a weight-average molecular mass of greater than 10⁶ and at least one alkaline hair-relaxing composition present in an amount that is sufficient to obtain permanent reshaping of keratin fibers treated with the composition.

The ready-to-use hair-relaxing composition may be prepared by mixing,

at least one alkaline hair-relaxing composition and

at least one composition chosen from

compositions comprising a dispersion of particles of at least one water-soluble polymer with a weight-average molecular mass of greater than 10⁶ in a saline aqueous solution, obtained by heterogeneous free-radical polymerization of water-soluble monomers with precipitation of the formed polymer and

compositions comprising an aqueous solution of at least one water-soluble polymer with a weight-average molecular mass of greater than 10⁶ obtained by heterogeneous free-radical polymerization of water-soluble monomers with precipitation of the formed polymer, wherein the various ingredients are those described in detail herein.

The pH of the ready-to-use hair-relaxing composition may range, for example, from 9 to 13 and, for example, from 10 to 13.

In another embodiment, the ready-to-use hair-relaxing composition may be prepared by mixing at least one alkaline hair-relaxing composition and at least one composition comprising a dispersion of water-soluble polymer particles, wherein the dilution of the saline dispersion medium with the aqueous at least one alkaline hair-relaxing composition has the effect of reducing the ionic strength and the total or partial dissolution of the water-soluble polymer particles.

The mixing proportion and the concentration of the dispersion or solution of the at least one water-soluble polymer may be chosen such that the at least one water-soluble polymer is present in an amount ranging from 0.01% to 10% by weight and, for example, from 0.05% to 2% by weight, relative to the total weight of the dispersion or the solution.

The ready-to-use hair-relaxing composition disclosed herein may also comprise at least one additive chosen from cosmetic active principles and formulation adjuvants commonly used in the field of haircare. For example, the at least one additive may be chosen from anionic, cationic, nonionic and amphoteric surfactants, fragrances, chelating agents, preserving agents, proteins, silicones, thickening polymers, plant, synthetic and mineral oils, waxes, buffers, UV-screening agents and hair dyes.

The ready-to-use composition may be provided in any form that allows easy application and spreading on the hair. Examples include emulsions, suspensions, solutions, gels, creams and pastes.

Other embodiments disclosed herein relate to processes for relaxing the hair comprising, separately, the at least one alkaline hair-relaxing composition and the at least one water-soluble polymer, or the ready-to-use hair-relaxing composition, described above. As disclosed herein, the protective effect on keratin fibers of the at least one water-soluble polymer is obtained either by simultaneous application of the at least one alkaline hair-relaxing composition and the at least one water-soluble polymer (one-step process) or successive application (two-step process).

The one-step hair-relaxing process comprises:

(A) applying at least one ready-to-use hair-relaxing composition disclosed herein to hair subjected to a mechanical constraint;

(B) leaving the at least one ready-to-use hair-relaxing composition on the hair for leave-in time that is sufficient to obtain permanent reshaping of the hair; and

(C) rinsing the hair.

This process may also comprise washing the hair with a neutralizing shampoo-wash.

As used herein, the term “mechanical restraint” includes any means that is suitable for keeping hair under tension, such as rollers and curlers, etc.

The two-step hair-relaxing process comprises,

(A) applying, to hair, at least one composition chosen from,

compositions comprising a dispersion of particles of at least one water-soluble polymer with a weight-average molecular mass of greater than 10⁶ in a saline aqueous solution, obtained by heterogeneous free-radical polymerization of water-soluble monomers with precipitation of the formed polymer and

compositions comprising an aqueous solution of at least one water-soluble polymer with a weight-average molecular mass of greater than 10⁶ obtained by heterogeneous free-radical polymerization of water-soluble monomers with precipitation of the formed polymer;

(B) optionally leaving the at least one composition on the hair for a leave-in time;

(C) optionally, rinsing the hair;

(D) applying at least one alkaline hair-relaxing composition to hair subjected to a mechanical constraint;

(E) leaving the at least one alkaline hair-relaxing composition on the hair for a leave-in time that is sufficient to obtain permanent reshaping of the hair; and

(F) rinsing the hair.

This process may further comprise washing the hair with neutralizing shampoo-wash.

The order of application of the at least one water-soluble polymer in dispersion and/or solution form (step A) and the at least one alkaline composition (step D) may be reversed, i.e., the application of the at least one alkaline hair-relaxing composition may precede the application of the at least one water-soluble polymer.

Some or all of the steps of these processes may be performed with application of heat.

The leave-in time that is sufficient to obtain permanent reshaping of the hair may range from 1 to 45 minutes, for example, from 1 to 30 minutes.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example is reported as precisely as possible. Any numerical value, however, inherently contain certain errors necessarily resulting from the standard deviation found in their respective testing measurements.

The various embodiments disclosed herein are illustrated with the aid of the example that follows.

### EXAMPLE 1

### Alkaline Hair-Relaxing Process—Demonstration of the Protective Effect of the High Molecular Weight Water-Soluble Polymers

The ready-to-use hair-relaxing alkaline compositions A and B below (pH=12) were applied to locks of natural hair, at a rate of 0.4 g per g of hair:

| | Composition A | | | |
|---|---|---|---|---|
| | (according to the | | Composition B | |
| | present disclosure) | | (comparative) | |
| NaOH | 1.99 | g | 1.99 | g |
| Water-soluble polymer* | 6.65 | g | — | |
| Water | qs 100 | g | qs 100 | g |

*Ultimer TX 11415, Ondeo

After a leave-in time of 20 minutes at room temperature, the locks were rinsed, drained, and then dried.

The chemical degradation of the locks were evaluated by measuring the porosity of the keratin fibers before and after alkaline treatment.

The porosity of the keratin fibers was measured by fixing at 37° C., over 2 minutes, 2-nitro-para-phenylenediamine at 0.25% in an ethanol/buffer mixture (10/90 volume ratio) at pH 10.

The results obtained are summarized in the following table:

| | Porosity |
|---|---|
| Untreated locks | 14 |
| Locks treated with solution A | 19 |
| Locks treated with solution B | 37 |

The porosity of the locks treated by simultaneous application of an alkaline composition and a high molecular weight water-soluble polymer (Composition A) was very similar to that of the locks before treatment, whereas the alkaline treatment in the absence of water-soluble polymer (Composition B) resulted in a large increase in porosity, which is a sign of substantial chemical degradation of the keratin fibers.

The locks of hair treated with Composition B also showed less satisfactory cosmetic properties, such as a coarser feel than that of the locks treated with Composition A.

What is claimed is:

1. A multi-component alkaline hair-relaxing agent comprising:
a first component (a) comprising at least one alkaline hair-relaxing composition, and
a second component (b) comprising at least one composition chosen from:
compositions comprising a dispersion of particles of at least one water-soluble polymer having a weight-average molecular mass of greater than 10⁶ in a saline aqueous solution, obtained by heterogeneous free-radical polymerization of water-soluble monomers with precipitation of the formed polymer and
compositions comprising an aqueous solution of at least one water-soluble polymer with a weight-average molecular mass of greater than 10⁶, obtained by heterogeneous free-radical polymerization of water-soluble monomers with precipitation of the formed polymer,
wherein the two components (a) and (b) are applied simultaneously or successively to the keratin fibers to be relaxed.

2. The multi-component alkaline hair-relaxing agent according to claim 1, wherein the at least one water-soluble polymer is chosen from polyelectrolytes.

3. The multi-component alkaline hair-relaxing agent according to claim 1, wherein the water-soluble monomers are chosen from at least one of cationic, anionic, and nonionic monomers comprising at least one ethylenic double bond.

4. The multi-component alkaline hair-relaxing agent according to claim 3, wherein the anionic monomers are chosen from acrylic acid, methacrylic acid, acrylamido-2-methylpropanesulphonic acid, and itaconic acid.

5. The multi-component alkaline hair-relaxing agent according to claim 3, wherein the nonionic monomers are chosen from acrylamide, methacrylamide, N-vinylformamide, N-vinylacetonamide, hydroxypropyl acrylate, and hydroxypropyl methacrylate.

6. The multi-component hair-relaxing agent according to claim 3, wherein the cationic monomers are chosen from di(C₁₋₄ alkyl)diallylammonium salts and the compounds of formula (I) 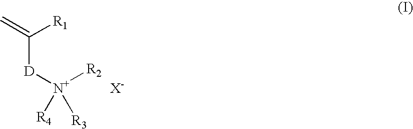
wherein:
R₁ is chosen from a hydrogen atom and a methyl group;
R₂ and R₃, which may be identical or different, are each chosen from a hydrogen atom and linear and branched C₁₋₄ alkyl groups;
R₄ is chosen from a hydrogen atom, linear and branched C₁₋₄ alkyl groups, and aryl groups;
D is chosen from the groups of the following formula, 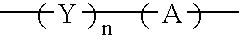
wherein:
Y is chosen from amide, ester, urethane and urea functional groups;
A is chosen from linear, branched and cyclic C₁₋₁₀ alkylene groups, which may be substituted or interrupted with at least one ring chosen from divalent aromatic and heteroaromatic rings, or which may be interrupted with at least one hetero atom chosen from O, N, S and P, and which may comprise at least one functional group chosen from ketone, amide, ester, urethane, and urea functional groups;
n is a number equal to 0 or 1; and
X⁻ is chosen from anionic counterions.

7. The multi-component alkaline hair-relaxing agent according to claim 6, wherein X⁻ is chosen from chloride and sulphate ions.

8. The multi-component alkaline hair-relaxing agent according to claim 1, wherein the at least one water-soluble polymer is polymerized from at least one cationic monomer of formula (I).

9. The multi-component alkaline hair-relaxing agent according to claim 8, wherein the at least one water-soluble polymer is polymerized from a monomer blend comprising from 0 to 30 mol % of acrylic acid, from 0 to 95.5 mol % of acrylamide, and from 0.5 mol % to 10 mol % of at least one cationic monomer of formula (I).

10. The multi-component alkaline hair-relaxing agent according to claim 8, wherein the at least one water-soluble polymer is polymerized from a monomer blend comprising acrylic acid and at least one cationic monomer of formula (I), wherein the number of moles of the at least one cationic monomer of formula (I) is greater than the number of moles of acrylic acid.

11. The multi-component alkaline hair-relaxing agent according to claim 8, wherein the at least one water-soluble polymer is polymerized from a monomer blend comprising 10 mol % of acryloyloxyethyldimethylbenzylammonium chloride and 90 mol % of acrylamide.

12. The multi-component alkaline hair-relaxing agent according to claim 8, wherein the at least one water-soluble polymer is polymerized from a monomer blend comprising 30 mol % of acryloyloxytrimethylammonium chloride, 50 mol % of acryloyloxyethyldimethylbenzylammonium chloride, and 20 mol % of acrylamide.

13. The multi-component alkaline hair-relaxing agent according to claim 8, wherein the at least one water-soluble polymer is polymerized from 10 mol % of acryloyloxyethyltrimethylammonium chloride and 90 mol % of acrylamide.

14. The multi-component alkaline hair-relaxing agent according to claim 8, wherein the at least one water-soluble polymer is polymerized from 30 mol % of diallyidimethylammonium chloride and 70 mol % of acrylamide.

15. The multi-component alkaline hair-relaxing agent according to claim 1, wherein the at least one water-soluble polymer is chosen from anionic polymers polymerized from 30 mol % of acrylic acid and 70 mol % of acrylamide.

16. The multi-component alkaline hair-relaxing agent according to claim 1, wherein the dispersion of particles of the at least one water-soluble polymer are present in an amount ranging from 0.01 % to 20% by weight, relative to the total weight of the dispersion.

17. The multi-component alkaline hair-relaxing agent according to claim 1, wherein the at least one water-soluble polymer in aqueous solution is present in an amount ranging from 0.01% to 20% by weight, relative to the total weight of the solution.

18. The multi-component alkaline hair-relaxing agent according to claim 1, wherein the at least one composition (b) further comprises at least one divalent anionic salt.

19. The multi-component alkaline hair-relaxing agent according to claim 18, wherein the at least one divalent anionic salt is chosen from ammonium sulphate, ammonium hydrogen sulphate, sodium sulphate, sodium hydrogen sulphate, magnesium sulphate, magnesium hydrogen sulphate, aluminium sulphate, and aluminium hydrogen sulphate.

20. The multi-component alkaline hair-relaxing agent according to claim 1, wherein the at least one alkaline hair-relaxing composition (a) has a pH ranging from 9 to 13.

21. The multi-component alkaline hair-relaxing agent according to claim 20, wherein the at least one alkaline hair-relaxing composition (a) has a pH ranging from 10 to 13.

22. The multi-component alkaline hair-relaxing agent according to claim 1, wherein the at least one alkaline hair-relaxing composition (a) is an aqueous solution comprising at least one hydroxide chosen from mineral and organic hydroxides, wherein the mineral and organic hydroxides are chosen from hydroxides of an alkali metal, hydroxides of an alkaline-earth metal, hydroxides of a transition metal, hydroxides of metals from groups III, IV, V and VI, lanthanide and actinide hydroxides, ammonium hydroxides, and guanidinium hydroxide.

23. A ready-to-use hair-relaxing composition comprising,
at least one water-soluble polymer with a weight-average molecular mass of greater than 10⁶ and
at least one alkaline hair-relaxing composition present in a concentration that is sufficient to permanently reshape keratin fibers treated with the ready-to-use hair-relaxing composition.

24. A ready-to-use hair-relaxing composition according to claim 23, wherein the ready-to-use hair-relaxing composition is obtained by mixing:
the at least one alkaline hair-relaxing composition and
at least one composition chosen from:
compositions comprising a dispersion of particles of at least one water-soluble polymer with a weight-average molecular mass of greater than 10⁶ in a saline aqueous solution, obtained by heterogeneous free-radical polymerization of water-soluble monomers with precipitation of the formed polymer and
compositions comprising an aqueous solution of at least one water-soluble polymer with a weight-average molecular mass of greater than 10⁶ obtained by heterogeneous free-radical polymerization of water-soluble monomers with precipitation of the formed polymer.

25. The ready-to-use hair-relaxing composition according to claim 23, wherein the ready-to-use hair-relaxing composition has a pH ranging from 9 to 13.

26. The ready-to-use hair-relaxing composition according to claim 25, wherein the ready-to-use hair-relaxing composition has a pH ranging from 10 to 13.

27. The ready-to-use hair-relaxing composition according to claim 23, wherein the at least one water-soluble polymer with a weight-average molecular mass of greater than 10⁶ is present in an amount ranging from 0.01 % to 10% by weight, relative to the total weight of the composition.

28. The ready-to-use hair-relaxing composition according to claim 27, wherein the at least one water-soluble polymer with a weight-average molecular mass of greater than 10⁶ is present in an amount ranging from 0.05% to 2% by weight, relative to the total weight of the composition.

29. A hair-relaxing process comprising:
(A) applying, to hair subjected to a mechanical constraint, at least one ready-to-use hair-relaxing composition comprising:
a first component (a) comprising at least one alkaline hair-relaxing composition, and
a second component (b) comprising at least one composition chosen from:
compositions comprising a dispersion of particles of at least one water-soluble polymer having a weight-average molecular mass of greater than 10⁶ in a saline aqueous solution, obtained by heterogeneous free-radical polymerization of water-soluble monomers with precipitation of the formed polymer and
compositions comprising an aqueous solution of at least one water-soluble polymer with a weight-average molecular mass of greater than 10⁶, obtained by heterogeneous free-radical polymerization of water-soluble monomers with precipitation of the formed polymer;
(B) leaving the at least one ready-to-use hair-relaxing composition on the hair for a leave-in time that is sufficient to obtain permanent reshaping of the hair; and
(C) rinsing the hair.

30. A hair-relaxing process comprising:
(A) applying to hair at least one composition chosen from:
compositions comprising a dispersion of particles of at least one water-soluble polymer with a weight-average molecular mass of greater than 10⁶ in a saline aqueous solution, obtained by heterogeneous free-radical polymerization of water-soluble monomers with precipitation of the formed polymer and
compositions comprising an aqueous solution of at least one water-soluble polymer with a weight-average molecular mass of greater than 10⁶ obtained by heterogeneous free-radical polymerization of water-soluble monomers with precipitation of the formed polymer;
(B) optionally leaving the at least one composition on the hair for a leave-in time;
(C) optionally, rinsing the hair;
(D) applying at least one alkaline hair-relaxing composition to hair subjected to a mechanical constraint;
(E) leaving the at least one alkaline hair-relaxing composition on the hair for a leave-in time that is sufficient to obtain permanent reshaping of the hair; and
(F) rinsing the hair.

31. The hair-relaxing process according to claim 30, wherein the order of steps (A) and (D) is reversed.

32. A multi-compartment kit for the alkaline relaxing of keratin fibers comprising,
at least one first compartment comprising a first component (a) comprising at least one alkaline hair-relaxing composition and
at least one second compartment comprising a second component (b) comprising at least one composition chosen from:
compositions comprising a dispersion of particles of at least one water-soluble polymer having a weight-average molecular mass of greater than 10⁶ in a saline aqueous solution, obtained by heterogeneous free-radical polymerization of water-soluble monomers with precipitation of the formed polymer and
compositions comprising an aqueous solution of at least one water-soluble polymer with a weight-average molecular mass of greater than 10⁶, obtained by heterogeneous free-radical polymerization of water-soluble monomers with precipitation of the formed polymer.

33. The multi-compartment kit according to claim 32, wherein the at least one water-soluble polymer is chosen from polyelectrolytes.
